(19) United States
(12) Patent Application Publication
(10) Pub. No.: US 2025/0163386 A1
(43) Pub. Date: May 22, 2025

(54) Cerebrovascular-Tumor-On-A-Chip

(71) Applicant: Ohio State Innovation Foundation, Columbus, OH (US)

(72) Inventor: Aleksander Skardal, Columbus, OH (US)

(21) Appl. No.: 18/838,519

(22) PCT Filed: Feb. 15, 2023
(86) PCT No.: PCT/US2023/062628

§ 371 Date: Aug. 14, 2024

Related U.S. Application Data
(60) Provisional application No. 63/268,025, filed on Feb. 15, 2022.

(51) Int. Cl. C12N 5/09; A61K 35/13; C12M 1/12; C12M 3/06; G01N 33/50

(52) U.S. Cl. CPC C12N 5/0693; A61K 35/13; C12M 23/16; C12M 25/14; G01N 33/5082; C12N 2503/04; C12N 2513/00; C12N 2533/70; C12N 2533/90

(57) ABSTRACT

A cerebrovascular unit tumor (CVU-T)-on-a-chip model that models both the BBB and tumor is described The model includes an ECM-mimicking hydrogel containing a variety of neural cell types and GBM tumor organoids derived from glioma patient tumor biospecimens embedded within the brain parenchyma of a CVU-T device. The model provided a physiologically relevant GBM tumor model including a corresponding microenvironment complete with a BBB component that is crucial in dictating drug bioavailability to the tumor.

### CROSS-REFERENCE TO RELATED APPLICATIONS

This application is claims the benefit of U.S. Provisional Application No. 63/268,025, filed Feb. 15, 2022, which application is incorporated herein by reference in its entirety.

### GOVERNMENT FUNDING

The present invention was made with government support under Grant No. R21CA229027 awarded by the National Institutes of Health. The US government has certain rights in this invention.

### FIELD

This invention relates to a Glioblastoma model for evaluating therapeutic treatments.

### SEQUENCE LISTING

The instant application contains a Sequence Listing which has been filed electronically in ASCII format and is hereby incorporated by reference in its entirety. Said ASCII copy, created on Feb. 15, 2023, is named 38801-189.xml and is 5506 bytes in size.

The following is a brief description of the Sequences: RGD (SEQ ID NO: 1), GFOGER (SEQ ID NO: 2), IKVAV (SEQ ID NO: 3), YIGSR (SEQ ID NO: 4), YGYYGDALR (SEQ ID NO: 5), FYFDLR (SEQ ID NO: 6)

### BACKGROUND

Glioblastoma (GBM), the most common primary brain tumor, has dismally low long-term survival. Despite aggressive treatment, involving surgical resection followed by chemo/radiotherapy, recurrence is nearly universal and the 5-year overall survival rate remains at only 5% with most tumors recurring within a year. Tumor recurrence in GBM patients is universal despite aggressive surgery and chemo/radiotherapy. Half a century of research has only increased average life expectancy by <3 months with just four new treatments gaining FDA approval. New therapies are urgently needed.

### SUMMARY

A bioengineered three-dimensional glioblastoma model for testing glioblastoma therapeutics is described. The model includes a blood brain barrier; a neural compartment, a brain parenchyma; and a tumor organoid, thus permitting both implants and crossing of the blood brain barrier to be evaluated. Use of the model then permits for evaluation of methods such treating glioblastoma in a subject.

Other methods, features and/or advantages is, or will become, apparent upon examination of the following figures and detailed description. It is intended that all such additional methods, features, and advantages be included within this description and be protected by the accompanying claims.

### BRIEF DESCRIPTION OF THE DRAWINGS

FIG. 1: Schematic describing the A) biological structure of the CVU-T, systemic TMZ delivery, and an optional implantable device for delivery of a therapeutic and/or adjuvant delivery.

FIG. 2A-2C: ECM composition modulates BBB transport. A) Our ECM hydrogel, comprised of HA, gelatin (or collagen), and PEG-based crosslinkers (to modulate elastic modulus), are supplemented with proteins or derivative peptides, such as lab-modified FN and LMN. Addition of FN/LMN results in B) robust EC layers expressing tight junction biomarkers (ZO-1), and C) control over molecular weight-based mass transport selectivity. Only with both FN and LMN present, can the BBB completely prevent 3-5 kDa MW FITC-dextran passage.

FIG. 3A-3C: Drug screening in PTOs from a responsive patient. A-C) PTO response to TMZ changes after cells have been cultured in 2D on tissue culture plastic. A) Freshly made organoids (no passaging—P0). B) PTOs made after 1 passage (P1) and C) 2 passages (P2). PTOs gain resistance to TMZ over time in 2D. *p<0.5 vs. control.

FIG. 4: HA-based ECM hydrogels preserve genomic profiles of GBM biospecimen-derived cells. RNAseq cluster analysis of originating tumor cells (Tu), organoids (Org), temozolomide-treated organoids (Org_T), and cells maintained in 2D tissue culture (Pla), from 6 human patients (A, C, E, F, G, and H). Tu, Org, and Org_T cluster together by patient, while Pla cultures cluster together, regardless of patient. Org/Org_T culture preserved the initial majority GBM subtype, while Pla promotes the mesenchymal subtype.

FIG. 5A-5D: CVU device design. a) The CVU uses 3 sets of corresponding media reservoirs to drive fluid flow. The device seen from above (scale bar—1 cm), and b) a cross section showing the tubular BBB organization within the ECM hydrogel and cells populating the 3D environment. Cell density is higher than depicted. c) DLP biofabrication is used to build multiple lumen-containing, high resolution CVU structures in parallel. The laser projector controls the patterning of crosslinking which changes as the build platform travels up out of the bioink volume. (i) Inset: A DLP bioprinted CVU structure. d) Flow of cells through the central lumen (L) within the parenchymal walls.

FIG. 6A-6C: A) τSTED images of murine intercalated disks show NaV1.5 sodium channels closely associating with Cx43 gap junctions. B) OBS3D analysis provides a quantitative picture of molecular organization in the form of bivariate histograms of sodium channel cluster mass vs. distance from Cx43 clusters (gap junctions). C) Enrichment ratios quantify the relative density of NaV1.5 channels near (<100 nm from cluster edges) Cx43 gap junctions [n=3 hearts/group, 5 images/heart; *p<0.05 vs. control, Wilcoxon's test].

FIG. 7A-7E: Representative STORM single molecule localization images showing NaV1.5 sodium channels and Cx43 gap junctions in murine hearts treated with A) vehicle (control) or B) VEGF (100 μg/ml, 60 minutes). STORM-RLA provides C, D) bivariate plots of NaV1.5 cluster density vs. distance from Cx43 clusters as well as E) simple indices such as the % of NaV1.5 molecules near Cx43 (<100 nm from cluster edge).

### DETAILED DESCRIPTION

A cerebrovascular unit tumor (CVU-T)-on-a-chip model that models both the BBB and tumor is described herein. The CVU-T advances current BBB models, which are largely planar, static, or both, to a tubular platform comprised of digital light processing (DLP) bioprinted brain ECM-mimicking hydrogel containing a variety of neural cell types. GBM tumor organoids, derived from glioma patient tumor biospecimens are embedded within the brain parenchyma of a CVU-T device, thus providing a physiologically relevant GBM tumor model including a corresponding microenvironment complete with a BBB component that is crucial in dictating drug bioavailability to the tumor. To bolster these efforts, in addition to a portfolio of chemical and biological assays, super-resolution microscopy-based technologies enable 3D mapping of individual cell types within CVU-Ts and the localization of 1) key protein targets relative to molecular landmarks and 2) fluorescently tagged drug compounds to directly validate drug delivery to the molecular target. In one aspect, the CVU-t-on-a-chip model is housed within a microfluidic device. The microfluidic device drives fluid flow throughout the model in parallel to the cells and blood brain barrier of the model. In one aspect, a drug, therapeutic, cell, or compound is introduced to the model through the fluid flow driven by the microfluidic device, thereby mimicking systemic administration of the drug, therapeutic, cell, or compound in vivo. In one aspect, the microfluidic device is fabricated by soft lithography or layering of laser cut components or 3D printed.

In one aspect, a bioengineered three-dimensional glioblastoma model for testing glioblastoma therapeutics is provided. The model including a blood brain barrier; a neural compartment, a brain parenchyma; and a tumor organoid. In some aspects, the tumor organoid is patient derived. In some aspects, the tumor organoid includes at least one of the following: astrocytes, pericytes, microglia, oligodendrocytes, neurons, and glioblastoma cells. In some aspects the tumor organoid includes all of the following: astrocytes, pericytes, microglia, oligodendrocytes, neurons, and glioblastoma cells.

### Definitions

As used herein and in the appended claims, the singular forms “a”, “and”, and “the” include plural referents unless the context clearly dictates otherwise. Thus, for example, reference to “a sample” also includes a plurality of such samples and reference to “the splicing regulator protein” includes reference to one or more protein molecules, and so forth.

As used herein, the term “about” refers to +/−1% deviation from the basic value.

As used herein, the term “tumor” refers to any neoplastic growth, proliferation or cell mass whether benign or malignant (cancerous), whether a primary site lesion or metastases.

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction of the undesirable proliferating cells with minimal destructive effects on normal cells. A subject at risk is a subject who has been determined to have an above-average risk that a subject will develop cancer, which can be determined, for example, through family history or the detection of genes causing a predisposition to developing cancer.

The term “subject,” as used herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos.

BBB refers to a blood brain barrier, or any highly selective semipermeable border of endothelial cells that prevents certain molecules that may be circulating blood from non-selectively crossing in the extracellular fluid of a central nervous system.

Unless defined otherwise, all technical and scientific terms used herein have the same meaning as commonly understood by one of ordinary skill in the art to which this invention belongs.

It will be appreciated the cerebrovascular unit-tumor on a chip model described herein has numerous applications beyond the glioblastoma model. For instance, a cerebrovascular unit-tumor on a chip model absent a tumor organoid, that is a cerebrovascular unit-on a chip model will be useful for assessing any brain related disorders including but not limited to MS, traumatic brain injury, Alzheimer's Disease, Parkinson's disease, ALS, cerebral palsy, concussion, dementias, LBD, epilepsy, headaches, migraine, stroke, Tourette Syndrome or sleep disorders.

### EXAMPLES

### Example 1—Development of a Cerebrovascular Unit Tumor (CVU-T)-On-a-Chip Model

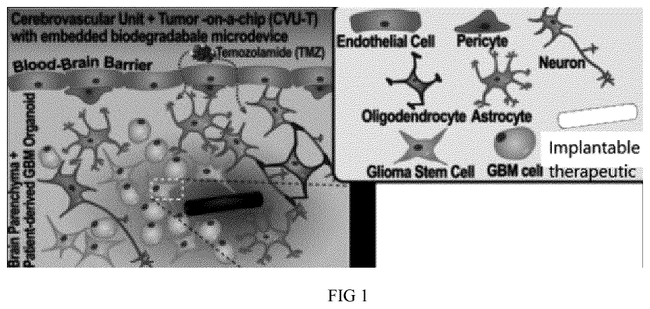

Referring to FIG. 1, a schematic describing the A) biological structure of the CVU-T, systemic therapeutic delivery, for example temozolaminde (TMZ), may be applied. An optional implantable device for delivery of a therapeutic and/or adjuvant delivery is also shown.

A 3D cellularized system models are still relatively rare. When combined with microfluidic device housings as described herein, these models address the shortcomings of previous planar and spheroid BBB models. We have advanced the complexity of the model system from simple spheroids to ECM hydrogel-supported tissue and tumor organoids—including patient-derived organoids that have undergone a variety of drug screening studies—and more complex “body-on-a-chip” platforms consisting of up to six organs/tissues on a chip. Unlike most other BBB platforms, our CVU enables a platform with multiple sampling points, compatibility with onboard sensing, while also using a 3D architecture of the CVU to mimic the blood-brain interface (FIG. 1).

We deploy a CVU-Tumor (CVU-T) tissue chip to test a combinatorial TMZ-adjuvant therapy, utilizing a bioengineered microcapsule-based drug delivery system to be implanted in the brain similar to standard-of-care placement of Gliadel wafers following surgical debulking of the tumor. The adjuvant-loaded microcapsules are implanted on the brain parenchymal side of the CVU-T, while systemically administering TMZ, and use super-resolution microscopy to validate payload delivery to mechanistic targets.

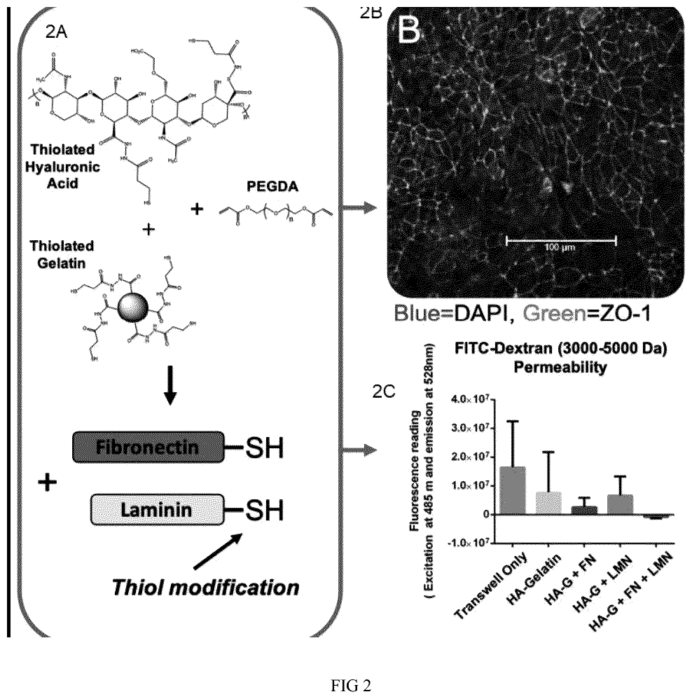

Referring to FIG. 2, a hydrogel formulation approach in which specific synthetically modified adhesion proteins, such as FN and LMN, can be covalently tethered to HA and collagen or gelatin base components (FIG. 2A). By modulating the ratios of the adhesion proteins, we could drive a tight brain microvascular endothelium monolayer, as visualized by zonula occludens-1 (ZO-1) immunofluorescence (FIG. 2B), and increased barrier function of this simplified BBB system. Barrier function is shown via trans-endothelial electrical resistance (TEER) sensing (FIG. 2C).

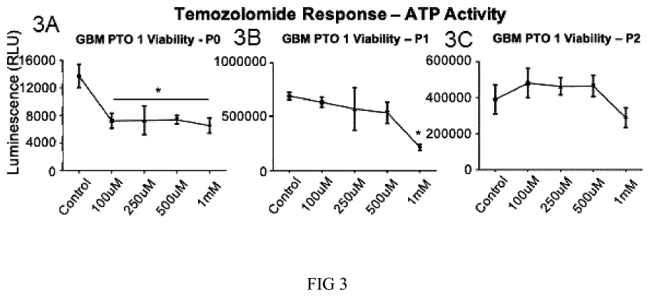

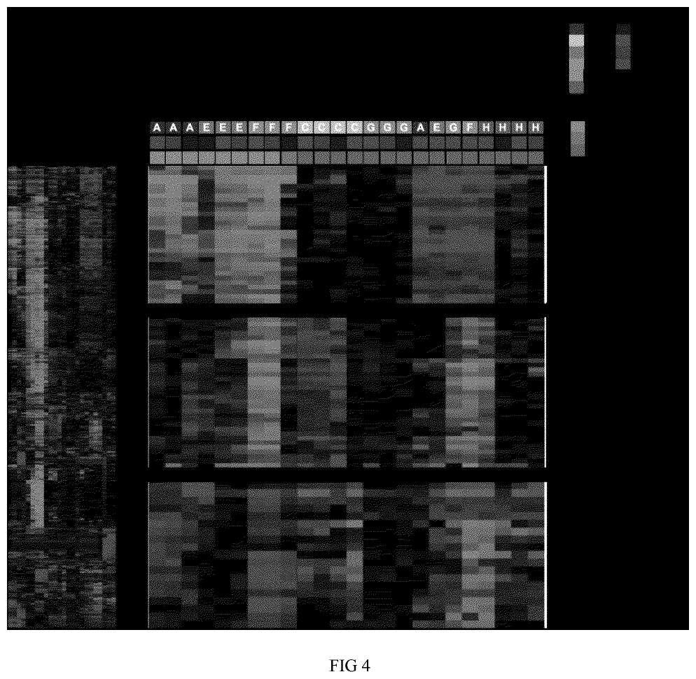

In parallel, we have established patient tumor organoids (PTOs) from a range of tumor types. These PTOs have been deployed in chemotherapy and immunotherapy studies. For example, FIG. 3 shows glioma PTO response to TMZ. Importantly, PTOs made directly from fresh tumor biospecimens showed TMZ sensitivity and tumor cell death. However, if the cells underwent just one or two passages in 2D culture, even if returned to 3D PTO form, they gained TMZ resistance. This suggests that the artificial nature of 2D culture drives a shift in phenotype and/or genotype. To further understand this shift, we analyzed RNA sequencing (RNAseq) data from biospecimens, 3D PTOs, TMZ-treated PTOs, and 2D cultured cells from 6 glioma patients. Cluster analysis of the 1000 most variably expressed genes resulted in clusters in which the original biospecimens, 3D PTOs, and TMZ-treated PTOs clustered together by patient, while in general the 2D cultured cell populations clustered together, while shifting to the mesenchymal subtype (FIG. 4). This RNAseq data strongly indicates that our 3D ECM hydrogel organoid system maintains the genetic profile of the originating tumor, and more accurately represent the diseases of individual patients.

### Example 2: Optimization and Characterization of a Locally Defined DLP Bioprinted CVU-Tumor Device

The CVU platform through modulation of ECM components and incorporation of primary and iPSC-derived neural cells and integrated GBM PTOs will fully realize the CVU-T system. Super-resolution microscopy techniques validate cell and tissue architecture and spatial resolution of molecular targets for drug studies in subsequent examples.

### Biofabrication of CVU-T

Neural cell cultures. Cells will be generated for each lineage using iPSCs (WiCell) ECs: iPSCs will be seeded and differentiated for 4 days in defined E6 medium. Impure immature cells are treated for 2 days with EC medium supplemented with bFGF and retinoic acid (RA). Mature brain microvessel endothelial cells (BMECs) are purified for 24 h in EC medium supplemented with bFGF and RA. 24 h after purification, barrier phenotype will be induced by treating cells with EC medium lacking bFGF and RA. Neural progenitor cells (NPC): This protocol builds on prior protocols. iPSC cultures are grown to 70% confluence then mTeSR1 is replaced with neural induction medium (Advanced DMEM: F-12 with Glutamax and 2% Neurobrew2l) with 1 μM IWR1 until day 5 and dual SMAD inhibition until day 10. On day 10, confluent cultures are treated with 10 μM Y-27632 for 1 hour before dissociation with Accutase. Cells are plated onto fresh laminin-coated plates, with a split ratio of 1:2 in Advanced DMEM: F-12 (with Glutamax). NPCs at this stage are strongly positive for PAX6, SOX2, FOXG1. Astrocyte-enriched cultures: NPCs are differentiated in planar culture in astrocyte medium (ScienCell). Astrocytes are defined by GFAP and S100β (cultures contain 90% S100β+ and 82% GFAP+ by flow cytometry). Cortical neuron differentiation: For differentiation, 65 NPCs are dissociated by Accutase and plated onto poly-D lysine- and LMN-coated dishes. Cortical neuronal differentiation medium consists of 1:1 DMEM/F12: Neurobasal, 1% N2 supplement, 1% B27 supplement, 1% L-glutamine 2 mM, 0.5 mg/ml bovine albumin fraction V, 55 μM β-mercaptoethanol and 8.6 mM glucose, with feeding 3-4 times per week. Neurons are assessed at 6 weeks in culture. Pericytes: Primary human brain pericytes are purchased directly from ScienCell and propagated in ScienCell Pericyte Medium.

Tumor cell acquisition and processing. We will investigate infiltrating gliomas with a focus on GBMs. Biospecimens will be obtained in cooperation the Ohio State University Comprehensive Cancer Center (OSUCCC) Tissue Procurement Core (TPC) (IRB protocol 2019C0196, Biorepository of Patient-Derived Tumor Models) and a new IRB protocol in preparation by Drs. Skardal and Elder. Dr. Elder will serve as facilitator between the clinical and laboratory research environments. We will obtain at least 45 patient biopsies (˜9/year). As a reference, over the past several years Dr. Skardal generated a pipeline for transferring biospecimens of various tumor types from the clinic to lab, resulting in over 30 glioma biospecimens being transferred into PTO-based studies in less than 1 year. The Skardal lab has biobanked cell suspensions and organoids from these biospecimens that can be used. All specimens are coded and deidentified, placed in RPMI (Roswell Park Memorial Institute) 1640 media, and then transferred through the TPC to the Skardal research team. Biospecimens are minced, washed, and incubated with collagenase and hyaluronidase to digest the ECM, as described. Portions will be preserved for histology and RNA extraction. Subsequent cell suspensions undergo dead cell removal and filtration prior to use.

CVU-T biofabrication: The conventional use of lithographically-defined PDMS elastomer for microfluidic devices is tedious due to reliance on expensive transparency masks, photolithography, serial casting, and precise layer alignment. However, this is necessary only for extraordinary resolution. Our 3D cell cultures enable the use of somewhat larger-scale, but considerably simpler thin, patterned adhesive films and poly(methyl methacrylate) (PMMA) layers that can be aligned and layered by folding and stacking to form microfluidic structures. This removes the need for a cleanroom or precise alignment and can be achieved by a computer-controlled laser cutter. TEER sensing will be integrated to these devices as previously described for continuous quantification. Notably, we will be employing a recent improvement to the physiological relevance of the BBB by moving from a planar model to using a tubular platform within a pump-optional microfluidic device (FIG. 5A-B) fabricated by 3D digital light processing (DLP, Cellink LUMEN X)) biofabrication (FIG. 5C), evolved from our extensive bioprinting experience. DLP printing will create a 3D ECM structure in which neural cells will be encapsulated, after which ECs will be seeded within the lumen. To do this, the tubular lumen structure will be “pulled” out of the hydrogel precursor reservoir with cells embedded, after which ECs will be seeded in the device to line the lumen walls. The PTO is embedded in the 3D volume of the CVU-T by first placing the PTO on predetermined locations on the reservoir surface. Thus, as the 3D lumen structure is formed, the PTO becomes embedded as the structure is formed.

ECM optimization for BBB function and characterization: ECM modulation. Thiolated HA and gelatin (Advanced Biomatrix) are dissolved 1% w/v, while a polyethylene glycol diacrylate (PEGDA, 3.4 kDa) crosslinker is dissolved at 2% w/v, in DI water containing 0.1% w/v photo-initiator (2-Hydroxy-4′-(2-hydroxyethoxy)-2-methylpropiophenone). These are mixed in a 2:2:1 ratio by volume and supplemented with thiolated FN, LMN, or collagen III and IV (modified in house through maleimide-thiol chemistry) at an end concentration of 0.25 μg/ml each. Pericytes, astrocytes, and neurons are suspended in the hydrogel precursor in density of 2×107 cells/ml each and are crosslinked by UV light (Is with intensity of 1 W/cm2). Modulation will primarily entail increases or decreases of the gelatin, FN, and LMN components by an order of magnitude, testing combinations of these 3 ECM components. Our aim is to maintain the HA structural component concentration to retain an elastic modulus in the range of brain tissue stiffness (400 to 2000 Pa). BBB integrity assays. Immunostaining with the following biomarkers will qualitatively assess the CVU construct: CD31, ZO-1, N-cadherin, GFAP, MAP2, GLUT1, beta-III-tubulin, and S100b.22 Macro-confocal microscopy (Leica TCI LSI) will be employed to provide visualization in 3D of interactions. Electrodes will be fabricated to fit CVU devices as in previous studies. Measurements are documented from readings using an Epithelial Volt/Ohm TEER Meter (WPIINC). Upon fabrication of the BBB, resistance will be low, increasing until reaching a plateau of EC confluency. Cell viability will be verified by LIVE/DEAD staining (Invitrogen). 70 kDa or 3 kDa FITC dextran in cell culture media will be added into the systemic device circulation. BBB transport will be determined by measuring fluorescence intensity of media aliquots in the brain fluid circuits. 3 kDa FITC dextran should pass through easily; 70 kDa should not. Only after increasing BBB permeability with histamine should the 70 kDa FITC dextran pass. Active transport will be assessed by studying activity of two efflux transport proteins commonly found in the BBB, p-glycoprotein (PgP) and multidrug resistant protein (MRP). Rhomadime 123 and H2DCFDA are fluorescent substrates of PgP and MRP respectively, so activity and directionality of the proteins can be easily quantified as previously described.

In some aspects, cell adhesion proteins such as laminin, fibronectin, collagen III, or collagen IV may serve as an additional extracellular matrix component to the CVU-T model. In some aspects the laminin, fibronectin, collagen III, or collagen IV, or other extracellular matrix protein is chemically modified with a functional group. The functional group provides for further customizing of the hyaluronic acid/gelatin/collagen components of the CVU models to fine tune the microenvironment of the model. In some aspects, an entire laminin, fibronectin, collagen III, or collagen IV, or other extracellular matrix protein is provided. In some aspects a peptide fragment of the laminin, fibronectin, collagen III, or collagen IV, or other extracellular matrix protein may be added as an additional extracellular matrix component. In some aspects the peptide fragment of the laminin, fibronectin, collagen III, or collagen IV, or other extracellular matrix protein is a sequence that provide a sequence a cell recognizes. In some aspects the peptide fragment may comprise the sequence: RGD (SEQ ID NO: 1), GFOGER (SEQ ID NO: 2), IKVAV (SEQ ID NO: 3), YIGSR (SEQ ID NO: 4), YGYYGDALR (SEQ ID NO: 5), FYFDLR (SEQ ID NO: 6). In some aspects the peptide fragment may consist of: RGD (SEQ ID NO: 1), GFOGER (SEQ ID NO: 2), IKVAV (SEQ ID NO: 3), YIGSR (SEQ ID NO: 4), YGYYGDALR (SEQ ID NO: 5), FYFDLR (SEQ ID NO: 6).

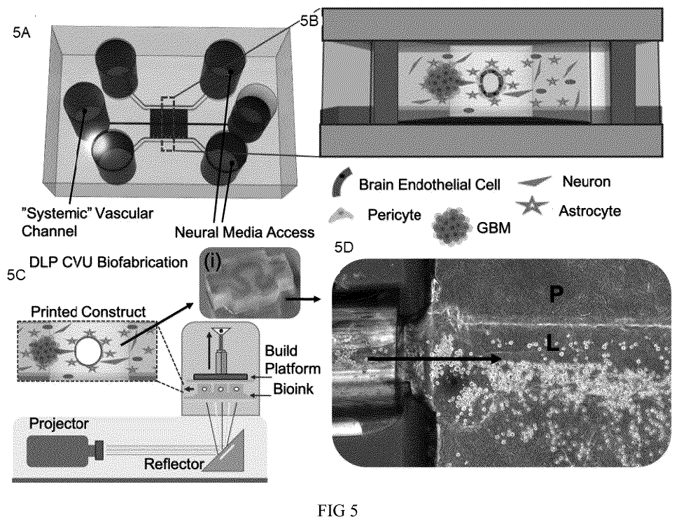

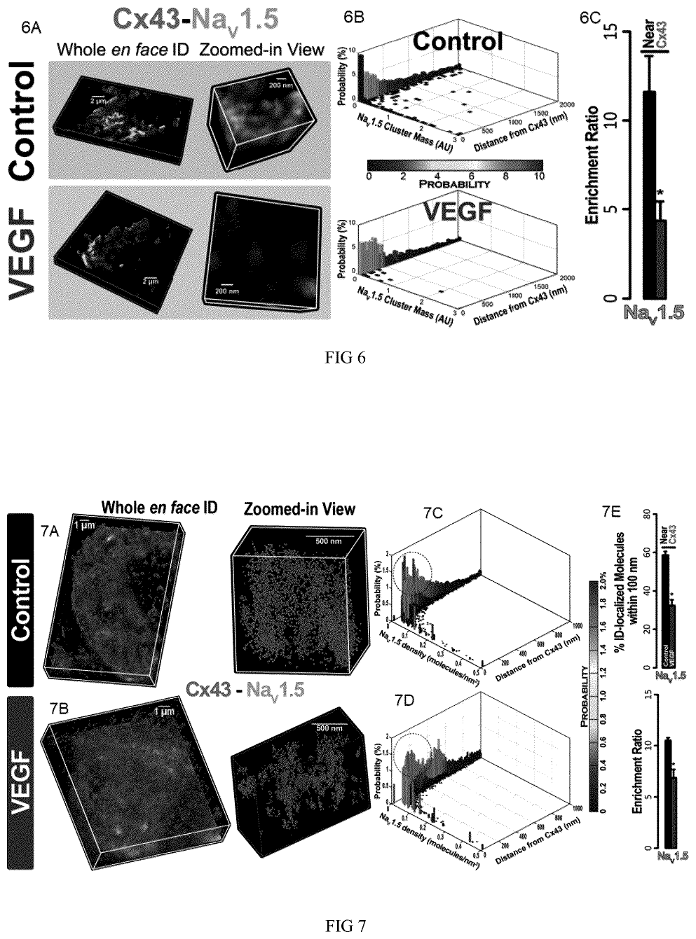

Quantitative assessment of CVU-T cellular and molecular structure: Spatial organization of cells within CVU-Ts will be assessed in 3D using sub-diffraction confocal microscopy (sDCI; FIG. 6), enabling simultaneous visualization of 4 spectral channels at 130 nm resolution. Resulting images will be analyzed using two approaches developed in the Veeraraghavan lab; Object-based Segmentation in 3D (OBS3D) and Morphological Object Localization (MOL) will quantify spatial organization of different cell types relative to each other as cumulative distribution functions of inter-cell type distances, enabling rich, quantitative assessment conducive to robust hypothesis testing (2-sample Kolmogorov-Smirnov tests to compare whole distributions, Wilcoxon's test to compare central tendencies). Additionally, these approaches will reveal spatial distribution of key proteins relative to cell-type markers and cellular landmarks (nuclei, cell periphery) within CVU-Ts. Proteins assessed may include Cx43, its scaffolding protein ZO-1 and mechanical junction proteins (N-cadherin, desmoglein 2, integrin β1). Furthermore, the spatial distribution of proteins identified at the cell surface and cell-cell contacts will be assessed at higher resolution with τSTED (fluorescence lifetime imaging-couplined STimulated Emission Depletion microscopy; simultaneous visualization of up to 2 spectral channels at <40 nm resolution with additional confocal channels; FIG. 6). MOL analysis of sDCI and τSTED images will quantify distribution of key cell types and proteins within CVU-Ts. Finally, the nanoscale distribution of therapeutic target Cx43 will be assessed relative to spatial landmark proteins (mechanical junction proteins identified at ell-cell contacts, particularly in GSCs) using STochastic Optical Reconstruction (STORM) microscopy (simultaneous localization of up to 2 protein species at 20 nm resolution). STORM (FIG. 7) will provide orthogonal validation of τSTED results plus additional information on molecular density. STORM single molecule localization data will be processed using machine learning-based cluster analysis [STORM-RLA] to quantitative 3D map of therapeutic targets within CVU-Ts, which are utilized in additional examples to assess treatment efficacy.

### Example 3: Patient-Derived GBM Tumor Organoids as an Assessment Tool for a Therapeutic Treatment Protocol

The CVU-T platform containing GBM PTOs is used to evaluate tumor cell killing efficacy of systemic temozolamide (TMZ) application to the model or alternatively the combinatorial effects of systemic TMZ application together with an implantable device for the sustained delivery of a therapeutic and/or adjuvant. The method using super-resolution microscopy to assess successful molecular targeting and efficacy of the drug treatment.

Combinatorial TMZ and/or adjuvant treatment in CVU-Ts: Combinatorial TMZ-adjuvant studies in CVU-Ts are initiated by incorporating the implantable device added in the parenchymal 3D CVU volume, embedded within the hydrogel upon DLP biofabrication. This implantable device may be a microcapsule, a nanoparticle or the like. For example, if a microcapsule is used, this is performed by placing microcapsules at predetermined locations on the DLP bioprinter build surface corresponding with the 3D digital CVU-T architecture. During bioprinting, the microcapsules become crosslinked into the parenchymal volume of the printed structures, as described for PTOs in Aim 1. Two top performing microcapsule designs from Aim 2 will be prepared and loaded with adjuvant. Blank microcapsules and therapeutic without encapsulation will be used as controls. Studies will use published techniques. At the termination of the studies, microcapsules will be retrieved from the CVU-Ts, and remaining therapeutic will be quantified by fluorescence after breaking capsules and washing with known volumes of PBS. This will enable calculation of therapeutic loading efficiency and percent released at each time point. Further, microcapsules will be imaged using SEM to evaluate changes in porosity and wall thickness to assess in vitro biodegradation and to check for any defects in the device. For drug treatment durations, we expect based on preliminary adjuvant release kinetics that 6 months of sustained therapeutic and/or adjuvant release is a realistic goal. Rather, our initial goal will be to perform 2, 4, 6, and 8 week adjuvant treatment regimens with corresponding analyses, potentially tuning TMZ administration. We have previously maintained both individual organoid and organ-on-a-chip systems, as well as 3- and 6-tissue organ-on-a-chip methods for at least 4 weeks with no major hurdles encountered. TMZ will be administered at 10 μM (expected; based on previous studies), delivered into the media reservoirs of the CVU-T devices, and be refreshed every 7 days.

Evaluation of therapeutic efficacy by traditional and super-resolution imaging assays Therapeutic bioactivity will be measured, but adapted to the CVU-T. LIVE/DEAD stains will be imaged on chip by confocal microscopy and cross-referencing the labeled PTO cells with the LIVE/DEAD calcein AM and ethidium homodimer-1 dyes. Co-localization with the PTO cell label positively identifies a GBM tumor cells, while lack of the label identifies a non-tumor neural cell of the CVU-T. If such cells fluoresce red, this would indicate off target toxicity, an unwanted side effect. Super-resolution imaging approaches from Aim 1 (sDCI, τSTED, STORM) will be used to acquire 3D Z-stacks of entire CVU-Ts. Fluorescently-labeled biomarkers distribution within CVU-Ts will be assessed in relation to its molecular target and other relevant proteins (N-cadherin, Desmoglein), cellular landmarks (cell periphery, nuclei), and cell type-specific markers. As described above, while imperfect, CD133, CD44, PDGFRA, and EGFR roughly correspond to the GSC, mesenchymal, proneural, and classic GBM molecular subtypes. While, new methods of subtyping are underway, because these more established subtypes do have specific genotypic and phenotypic differences, we will assess how treatment impacts each subpopulation. OBS3D and MOl analysis of sDCI, τSTED images will enable the assessment of therapeutic and/or adjuvant distribution throughout the volume of CVU-Ts in relation to cell-type markers and key cell-cell contact proteins. This will reveal how the therapy targets individual GBM subpopulations and validate peptide delivery to target-rich cell membrane/cell-cell contact niches within them.

Tumor organoid maintenance of genomic profile: RNAseq and hierarchical cluster analysis will be performed as described in FIG. 4 on tumor regions extracted from CVU-Ts in a verification step to determine the PTOs' and CVU-Ts' maintenance of the genomic profile of the originating tumor. Because the PTOs are labeled, CVU-Ts can be treated like tissue biospecimens and be dissociated w/ collagenase/hyaluronidase and dispase as described in Aim 1. Labeled glioma cells will be isolated by FACS for RNAseq and compared with that of the originating biospecimen. We will isolate RNA (Qiagen RNeasy) and RNAseq will be performed for the populations in question (OSUCCC Genomic Shared Resource), with dataset alignment performed at the Ohio Supercomputer Center, and analyzed with our bioinformatics core. In particular, we are interested relative expression changes of genes associated with the GSC, mesenchymal, proneural, and classic subpopulations (e.g., CD133, CD44, PDGFRA, EGFR). For more breadth, we will also perform hierarchical cluster analysis (R, R-Project) of the 1000 most variably expressed genes to verify continued maintenance of the original tumor genomic profiles in the CVU-Ts (i.e., FIG. 4).

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

1. An in vitro bioengineered three-dimensional glioblastoma model, the model comprising:
a blood brain barrier,
a neural compartment,
a brain parenchyma, and
a tumor organoid.

2. The model of claim 1, further comprising an extracellular matrix hydrogel.

3. The model of claim 1, further comprising an implantable device for release of a therapeutic treatment and/or adjuvant to the tumor organoid.

4. The model of claim 1, the tumor organoid being a patient derived tumor biospecimen.

5. The model of claim 1, the tumor organoid comprising one or more cells that is are astrocytes, pericytes, microglia, oligodendrocytes, neurons, or glioblastoma cells.

6. The model of claim 1, the tumor organoid comprising one or more of the cells are encapsulated in an extracellular matrix-based hydrogel, the hydrogel comprising:
hyaluronic acid, collagen, or gelatin,
wherein the hyaluronic acid, collagen, or gelatin are chemically modified to enable covalent crosslinking to one another to form a hydrogel, and
wherein additional extracellular matrix proteins or polysaccharides are covalently bound to the hyaluronic acid, collagen, or gelatin.

7. The model of claim 6, wherein the additional extracellular matrix protein comprises a laminin, a fibronectin, a collagen III, a collagen IV, a peptide fragment comprising RGD (SEQ ID NO: 1), a peptide fragment comprising GFOGER (SEQ ID NO: 2), a peptide fragment comprising IKVAV (SEQ ID NO: 3), a peptide fragment comprising YIGSR (SEQ ID NO: 4), a peptide fragment comprising YGYYGDALR (SEQ ID NO: 5), or a peptide fragment comprising FYFDLR (SEQ ID NO: 6).

8. The model of claim 1, wherein a blood brain barrier is evaluated by the presence of a tight junction biomarker or evidence of transport selectivity based on molecular weight of a substance.

9. The model of claim 1, wherein the tumor organoid cells are not subject to cellular passage so that the tumor organoid of the three-dimensional glioblastoma model retains the characteristics of the original tumor organoid.

10. The model of claim 1, wherein the model is housed within a microfluidic device.

11. The model of claim 9, wherein the microfluidic devices drives fluid flow throughout the model in parallel to the cells and blood brain barrier of the model.

12. The method of claim 10, wherein a drug, therapeutic, cell, or compound is introduced to the model through the fluid flow driven by the microfluidic device, thereby mimicking systemic administration of the drug, therapeutic, cell, or compound in vivo.

13. The model of claim 1, wherein the microfluidic device is fabricated by soft lithography or layering of laser cut components or 3D printed.

14. A method of assessing the effectiveness of a treatment for glioblastoma comprising:
providing a three-dimensional glioblastoma model, the model comprising:
a blood brain barrier,
a neural compartment,
a brain parenchyma, and
a tumor organoid;
applying to the three dimensional glioblastoma model a treatment for glioblastoma;
measuring a parameter including the concentration of therapeutic delivered to the tumor organoid, the concentration of therapeutic delivered across a blood bran barrier, a decrease in the tumor organoid size, or an alteration in the cells surrounding the tumor organoid,
wherein a change in a parameter as compared to an untreated three-dimensional glioblastoma model permits evaluation of the effectiveness of a treatment for glioblastoma

15. The method of claim 13, wherein the three-dimensional glioblastoma model is patient specific, the tumor organoid being patient derived and the effectiveness of a treatment for glioblastoma is specific for the patient.

16. An in vitro bioengineered three-dimensional brain cerebrovascular model, the model comprising:
a blood brain barrier,
a neural compartment, and
a brain parenchyma.

17. The model of claim 15, further comprising an extracellular matrix hydrogel.

18. The model of claim 15, further comprising one or more cells that is are astrocytes, pericytes, microglia, oligodendrocytes, neurons, or glioblastoma cells.

19. The model of claim 15, the tumor organoid comprising one or more of the cells are encapsulated in an extracellular matrix-based hydrogel, the hydrogel comprising:
hyaluronic acid, collagen, or gelatin,
wherein the hyaluronic acid, collagen, or gelatin are chemically modified to enable covalent crosslinking to one another to form a hydrogel, and
wherein additional extracellular matrix proteins or polysaccharides are covalently bound to the hyaluronic acid, collagen, or gelatin and the extracellular matrix comprises a laminin, a fibronectin, a collagen III, a collagen IV, a peptide fragment comprising RGD (SEQ ID NO: 1), a peptide fragment comprising GFOGER (SEQ ID NO: 2), a peptide fragment comprising IKVAV (SEQ ID NO: 3), a peptide fragment comprising YIGSR (SEQ ID NO: 4), a peptide fragment comprising YGYYGDALR (SEQ ID NO: 5), or a peptide fragment comprising FYFDLR (SEQ ID NO: 6).

20. The model of claim 18, wherein the model is housed within a microfluidic device.


SEQUENCE LISTING (6 sequences)

SEQ ID NO: 1 - skipped

SEQ ID NO: 2 - protein, 6 aa, synthetic construct
GFOGER

SEQ ID NO: 3 - protein, 5 aa, synthetic construct
IKVAV

SEQ ID NO: 4 - protein, 5 aa, synthetic construct
YIGSR

SEQ ID NO: 5 - protein, 9 aa, synthetic construct
YGYYGDALR

SEQ ID NO: 6 - protein, 6 aa, synthetic construct
FYFDLR